(19) United States
(12) Patent Application Publication
(10) Pub. No.: US 2003/0181807 A1
(43) Pub. Date: Sep. 25, 2003

(54) Method, device and system for implanting a shunt

(76) Inventors: Kieran P. Murphy, Baltimore, MD (US); Michael Williams, Baltimore, MD (US); Danielle Rigamonti, Baltimore, MD (US)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/361,692

(22) Filed: Feb. 11, 2003

Related U.S. Application Data
(60) Provisional application No. 60/366,529, filed on Mar. 25, 2002.

(51) Int. Cl. A61B 5/05

(52) U.S. Cl. 600/411; 600/424

(57) ABSTRACT

One embodiment of the invention provides a trocar for use in implanting a shunt. The trocar is made from a material with sufficient rigidity to travel through human tissue, and also made from a material that has a radioopacity that substantially preseves the appearance of the trocar when the trocar is viewed under a substantially real-time medical imaging system, such as the Toshiba Acquillon.

### PRIORITY CLAIM

The present application claims priority from U.S. Provisional Patent Application No. 60/366,529 filed Mar. 25, 2002, the contents of which are incorporated herein by reference.

### FIELD OF THE INVENTION

The present invention relates generally to surgery under image guided navigation and more particularly relates to a method, device and system for surgical implantation of a shunt or the like under image guidance.

### BACKGROUND OF THE INVENTION

There are a variety of conditions that are treatable through surgery, wherein the surgeon navigates through the body of the patient. For example, intracranial hemorrhage (ICH) is one of the most serious types of stroke. More than 700,000 new strokes occur in the United States yearly. 20% of these are hemorraghic with more then half of them presenting with ICH. The 30 day mortality is 68% (Naff et al. Stroke 2000;31:841-847). Another type of condition that is treatable through surgery is hydrocephalus and related conditions. Temporary CSF drainage shunts are well known and used broadly to treat patients with acute hydrocephalus secondary to bleed, stroke, as well as tumour or any obstruction to CSF drainage. Prior art shunt devices are simple single lumen silicon tubes with perforations near their tip. They are introduced over a rigid canula without image guidance. This is done at the bedside, by a neurosurgeon, based on the palpation of boney land marks of the skull. A thrombolytic drug can then be injected via this tube into the ventricle with the intent of dissolving the clot and removing the mass effect and the obstruction to CSF flow. The mortality of these brain hemorrhages can be reduced to 22% by this technique, as discussed in Naff et al Stroke 2000;31:841-847, (“Naff”) Montes et al. Stroke 2000;31:834-840, (“Montes”). The contents of both these documents are incorporated herein by reference.

By and large, these prior art shunts have been unchanged in design for 30years. In simple terms, such ventriculostomies, (also known as shunts), typically have an inlet located in the patient's brain, and an outlet outside the skull which can accept and expel the excess fluid. A detailed discussion of prior art CSF shunts can be found in Drake et al, The Shunt Book, © 1995 Blackwell Science, Inc. Massachusetts, (“Drake”) the contents of which are incorporated herein by reference.

More particularly, shunts used ventriculostomies are designed to drain CSF from the brain. Ventriculoperitoneal shunts (“VP shunts”) are used in a variety of medical conditions and are implanted in both young and old patients. Certain configurations of prior art VP shunts can include a ventricular catheter, a flow-valve that can be changed by simple hydrostatic pressure or a switching mechanism, and a CSF drainage tube for draining the excess CSF. A hole is drilled in the skull, and the ventricular catheter stiffened by an introducing stylet is pushed through the brain until CSF returns down the central lumen. The major complications from these and other prior art shunts include stroke, bleeding, damage to adjacent brain, infection, obstruction, disconnection, under draining, and over draining, all of which can lead to serious injury and even death. The symptoms of shunt failure and malfunction are nonspecific and include fever, nausea, vomiting, irritability and malaise. A patient presenting with such symptoms warrants a thorough radiological, laboratory, and occasionally a surgical evaluation. As known to those of skill in the art, insertion of VP shunts requires a highly skilled neurosurgeon, but once inserted, such shunts are frequently prone to failure and need revision.

Recent advances in the art of surgery have attempted to overcome some disadvantages of older shunts. For example, the use of telemetry is now contemplated, as discussed in Miyake H. et al., “A new ventriculpertoneal shunt with a telemetric intracranial pressure sensor: clinical experience in 94 patients with hydrocephalus”, Neurosurgery, May 1997; 40(5): 931-5 and Munshi H.., “Intraventricular pressure dynamics in patients with ventriculopleural shunts: a telemetric study”, Pedatr Neursurg,Feb 1998; 28(2): 67-9 Despite the fact that Miyake and Munshi teach the use of telemetrics with shunts, the shunts taught therein are still prone to failure due to infection, blockages and other difficulties, such that failures of such shunts can still require complete replacement of the shunt.

In addition to the foregoing limitations, generally, prior art shunt tubes and their delivery systems are straight. The location of the tip after placement was based on the resistance it encountered during delivery. The tip or portions of these blindly introduced catheters often did not enter the ventricle at all. Because the systems were straight the operator had little control of the delivery other then reintroduction of the entire system. The repositioning of the catheter requires repeat passage through the intervening brain with the risk of bleeding, stroke and the introduction of infection.

Of interest to those practicing in the area of VP shunts, there have been recent advances in image guidance that allow real time CT guidance, MRI guidance or the like, for navigating during procedures. For example, CT scanners such as the Toshiba Acquillion multi detector are capable of generating images in 3 different areas at frame rates of 13 frames a second. However, despite the advances in shunt technology and imaging technology, prior art shunt technology have certain limitations when inserted under image guidance and, there still remains a significant likelihood of patient death during treatment for the above-identified conditions, and it is generally believed that further advances to both shunt technology and surgical navigation are desirable.

### SUMMARY OF THE INVENTION

It is therefore an object of the invention to provide a method, device and system for implanting a shunt or the like that obviates or mitigates at least one of the above-identified disadvantages of the prior art.

In a first aspect of the invention there is provided a method and device to use imaging to provide guidance for catheter delivery into the ventricle, and corresponding navigation within that space. The inserted catheter can then be used for the dissolution and removal of blood secondary to hemorrhage or installation of drugs to treat tumor infection or other disease.

In a second aspect of the invention there is provided a method for implanting a shunt under guidance of a substantially real-time imaging display system comprising the steps of:

inserting a trocar comprised of a stylet and a sleeve, the trocar having a rigidity to travel to a target area in mammalian tissue and having a radioopacity that substantially preserves the trocar's appearance when the trocar is viewed under the imaging display system; and,

removing the stylet from the trocar and leaving the trocar inside the target area.

In a particular implementation of the second aspect, the method further comprises the additional steps of:

inserting a wire into the sleeve, the wire having a rigidity to travel through the sleeve and a radioopacity that substantially preserves the wire's appearance when the wire is viewed under the system, the insertion terminating when a distal tip thereof resides in the target area; and,

removing the sleeve of the trocar and leaving the wire in the target area.

passing a catheter over the wire until the catheter reaches a desired location in the target area; and,

removing the wire and leaving the catheter therein.

After the passing step, there can be the additional step of manipulating, under the guidance of the imaging system, the wire and the catheter until the wire and the catheter are in a desired position within the target area.

In a third aspect of the invention there is provided a medical device for use under a guidance of a substantially real-time imaging display system comprising:

a body for travelling through mammallian tissue and having a rigidity to travel therethrough to a target area therein and having a radioopacity that substantially preserves the body's appearance when the body is viewed under the image guidance system.

In a particular implementation of the third aspect, the mammallian tissue is human tissue.

In a particular implementation of the third aspect, the device is a trocar having a sleeve and a stylet.

The target area can be selected from the group consisting of the ventricular system, intraparachymal space, subarachnoid space, subdural space. The target area can be a clot in the ventricular system. The target area can be an excess of CSF fluid in the ventricular system

The imaging system can be selected from the group consisting of a real-time CT machine, and a real time MR machine. One suitable real-time CT machine is the Toshiba Acquillion.

The medical device can either be coated-with or made-from a material that is hydrophilic.

The medical device can be coated-with an infection-resistant material.

The medical device can be a wire for passing down a sleeve resting in the target area. The wire has a resiliently deformable loop on the distal end of the wire, the loop having a straightened position for passing down the sleeve and a looped position for engaging with the target area.

In a fourth aspect of the invention there is provided a method for implanting a shunt comprising the steps of:

inserting a trocar guided under a substantially real-time imaging system into a target area of the skull;

removing the stylet from the trocar and leaving a sleeve thereof inside the ventricular system;

inserting a radioopaque wire into the sleeve using the display until a distal tip thereof resides in a desired location of the ventricular system;

removing the sleeve and leaving the wire in the ventricular system;

passing a catheter over the wire until the catheter reaches a desired location in the ventricular system; and,

removing the wire and leaving the catheter therein.

### BRIEF DESCRIPTION OF THE DRAWINGS

Preferred embodiments of the invention will now be discussed, by way of example only, with reference to the attached Figures, in which:

FIG. 1 is a perspective exploded view of a trocar for implanting a shunt through a previously drilled burr hole in the skull of a patient's head;

FIG. 2 is a representation of a frame of an axial image from a CT scan of the patient÷s skull in FIG. 1 wherein the trocar is being intraventricularly inserted into head;

FIG. 3 is a sectional view through lines III-III of FIG. 2 of the patient's skull but showing the stylet being removed from the sleeve of the trocar;

FIG. 4 is the sectional view of FIG. 3 showing a wire being inserted into the sleeve;

FIG. 5 is the sectional view of FIG. 4 showing the wire being inserted through the sleeve;

FIG. 6 is the sectional view of FIG. 5 showing the wire exiting through the distal tip of the sleeve;

FIG. 7 is the sectional view of FIG. 6 showing the distal tip of the wire looping and abutting against the clot inside the patient's head;

FIG. 8 is the sectional view of FIG. 7 showing the sleeve being removed;

FIG. 9 is the sectional view of FIG. 8 showing a catheter being passed over the wire;

FIG. 10 is the secitional view of FIG. 9 showing the wire being pulled out of the catheter;

FIG. 11 is the sectional view of FIG. 10 showing the catheter remaining inside the patient's head;

FIG. 12 is a front view of a trocar in accordance with another embodiment of the invention;

FIG. 13 shows the trocar of FIG. 12 inserted into a patient's skull;

FIG. 14 shows the trocar of FIG. 12 with the needle of the trocar being removed from the trocar;

FIG. 15 shows a wire being inserted into a clot inside a ventricular system in accordance with another embodiment of the invention;

FIG. 16 shows the wire of FIG. 15 being manipulated so that its end creates a hockey-stick shape;

FIG. 17 shows the wire of FIG. 16 having a cathether passed thereover;

FIG. 18 shows the wire of FIG. 17 wherein the end of the catheter forms a hockey-shaped tip complementary to the wire;

FIG. 19 shows the cathether of FIG. 18 and with the wire removed therefrom; and,

FIG. 20 shows the wire and cathether of FIG. 18 rotated within the clot.

### DETAILED DESCRIPTION OF THE INVENTION

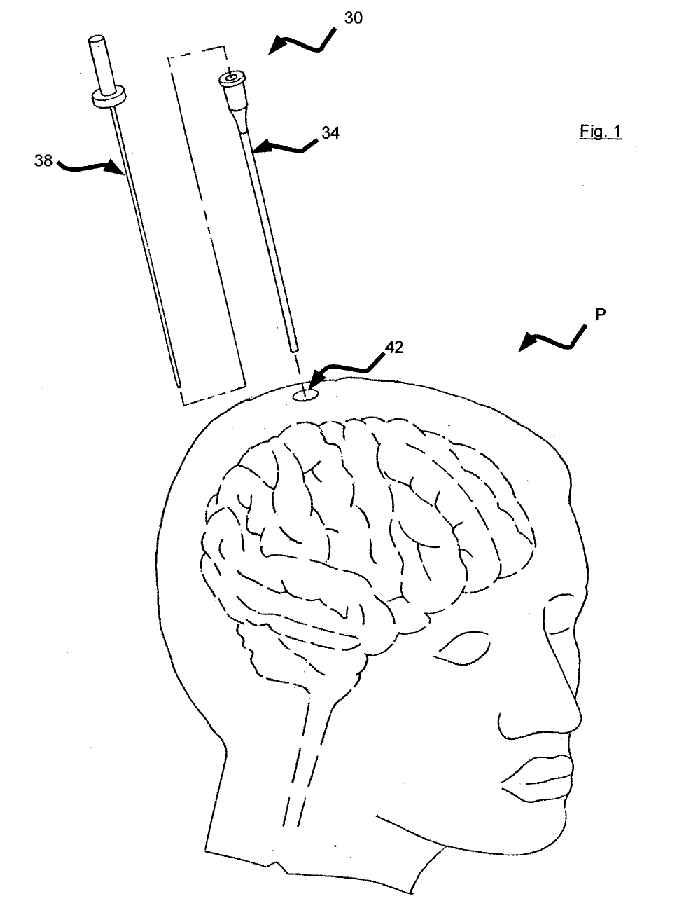

Referring now to FIG. 1, in an embodiment of the invention there is provided a trocar for introducing a catheter for a shunt or the like, indicated generally at 30. Trocar 30 comprises a sleeve 34 that coaxially surrounds a stylet 38 when stylet is inserted into sleeve 34. When stylet 38 is inserted into sleeve 34, the resulting tip of trocar 30 is chosen to have any suitable or desired shape for piercing through the tissue of a patient P when trocar is inserted therin. In a presently preferred embodiment, the tip is a round arrow-head, for travelling to through the patient's brain and into the ventricular system, via a previously drilled burr hole 42 in patient P's skull.

Trocar 30 (i.e. Its constituent parts sleeve 34 and stylet 38) is made from a material that is hard enough and/or rigid enough to effect the desired piercing and travelling, but is also made from a material with a radioopacity density such that trocar 30 creates a reduced level of artifacts when placed in an imaging beam, such as the imaging beam of a CT machine. In general, trocar 30 has a radioopacity that substantially preserves trocar 30's appearance when viewed under the imaging beam. Further a presently preferred CT machine for use in conjunction with trocar 30 is the Toshiba Acquillion, or any other CT machine capable of generating a sufficiently high frame rate to allow real time image generation during a surgical navigation through a patient. Thus, the material of trocar 30 is chosen to have reduced artifacts when used in conjunction with the desired imaging beam. As known to those of skill in the art, such density can be measured in terms of “Hounsfield units”, wherein a lower Hounsfield unit represents a reduced the artifact effects when placed under an imaging beam. Suitable materials can include, but are not limited to, certain plastics, carbon fiber and Inconel metals.

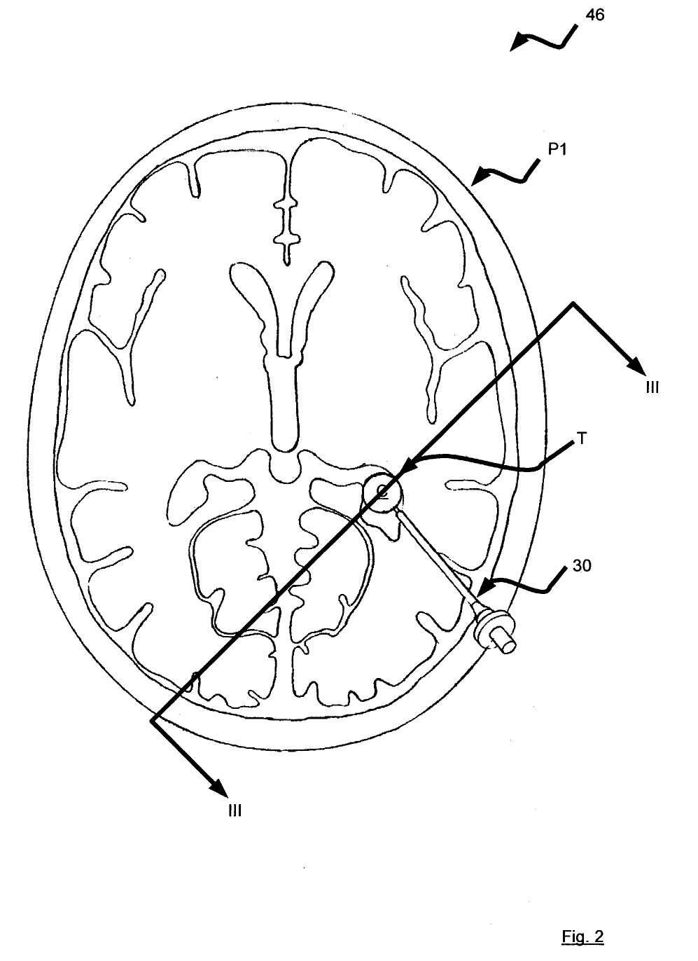

In another embodiment of the invention, there is provided a method for implanting a catheter for a shunt or the like. It is presently preferred that the method is performed using a high-image rate device, such as a high-image rate CT machine, such as the Toshiba Acquillion or the like, to provide substantially real-time images of the area of patient P being exposed to the imaging beam. In order to perform the method, patient P is placed in the beam of the CT Machine and prepped in the usual manner. Referring now to FIG. 2, an exemplary single frame of an image of patient P generated by a beam of the CT machine (or other suitable imaging device) is indicated generally at 46. Frame 46 includes an axial image of patient P's skull, indicated at P1. Frame 46 shows a target area T that is to be treated using the method of the present embodiment. In the present embodiment, target area T is located inside a ventricle of patient P, but other target areas can be selected, such as the intraparenchymal space to treat an acute intraparenchymal hemorrhage or other condition associated therewith. Other target areas could include the subarachnoid space or the subdural space, or such other target areas as will now occur to those of skill in the art. In the present embodiment, it is assumed that there is a clot C located at target area T, and the clot C will be the condition that is being treated according to the method, and thus more particularly target T is at a suitable point for inserting a shunt drainage catheter that can be used to drain clot C.

Frame 46 also shows trocar 30 overlayed thereon, with trocar 30 having been inserted into patient P's skull, and with the tip of trocar 30 being positioned inside a target area T of frame 46. The representation of the entirety of trocar 30 is for illustrative purposes, and it is to be understood that typically, only a portion of the tip of of trocar 30 will be shown within frame 46 as part of the axial image being generated by the CT machine. However, it is contemplated that at least some movement of trocar 30 can be effected to put trocar 30 “out-of-plane” with the axial view of frame 46, thereby showing at least some of the length of trocar 30. Thus, according to the performance of the method of the present embodiment, trocar 30 is inserted through burr hole 42 in the patient's skull, until trocar 30 reaches a target area T.

Because trocar 30 is selected from a material with suitable radioopacity, it does not create (or only creates suitably reduced) artifacts on the display of the CT Machine during the real-time displaying of the insertion of trocar 30. While a rate of image display in “real-time”, such as about 13 frames per second (“fps”) or greater is presently preferred. Other rates can also be chosen, such as greater than about 20 fps, or greater than about 25 fps, or greater than about 30 fps. It is to be understood that any or other suitable speed to allow an appropriate level of accuracy and/or sufficient level of information during navigation through patient P's head can be used. Thus, using the high-speed imaging capabilities of the CT Machine in conjunction with trocar 30 having a sufficient hardness to pierce through head while having low enough radioopacity to produce reduced amounts of artifacts on the display of the CT Machine, the surgeon is able to safely introduce trocar 30 into the ventricular system of head.

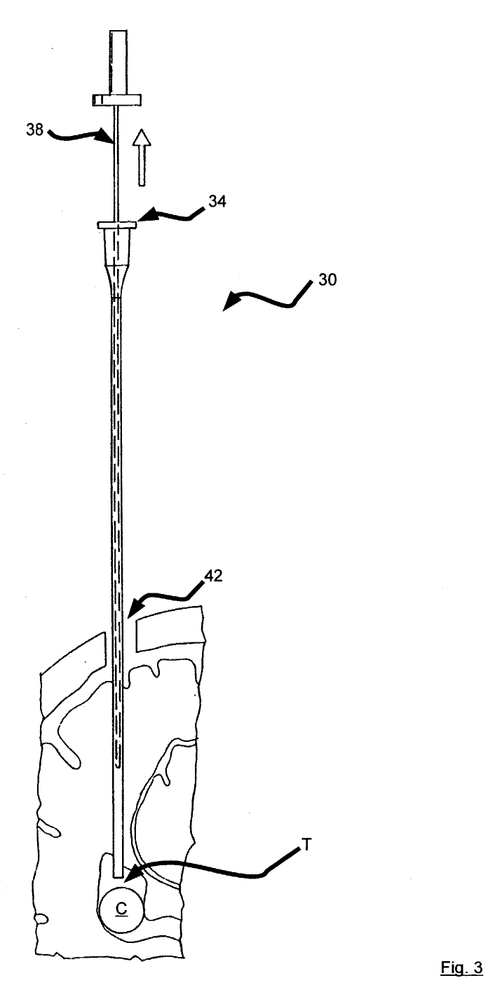

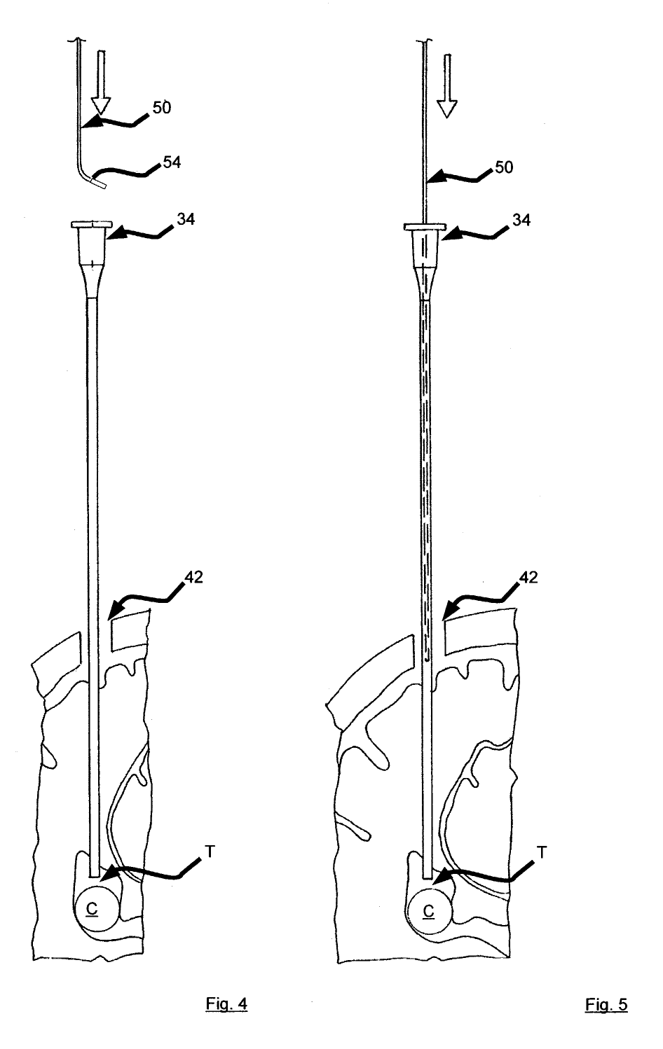

Next, as shown in FIG. 3, stylet 38 is withdrawn from sleeve 34, while stylet 38 is held in place. Next,as seen in FIGS. 4 and 5, a resiliently bendable wire 50 is passed down sleeve 34 towards target area T. Wire 50 is made from a material that is radioopaque, but also does not create (or only creates suitably reduced) artifacts on the display of the CT machine being used. As best seen in FIG. 4, wire 50 preferably has a hockey-stick shaped bend 54 at its distal end, typically at about a forty-five degree angle (but other suitable angles will occur to those of skill in the art). As best seen in FIG. 5, the bend 54 can be straightened so that can pass through the lumen of sleeve 34.

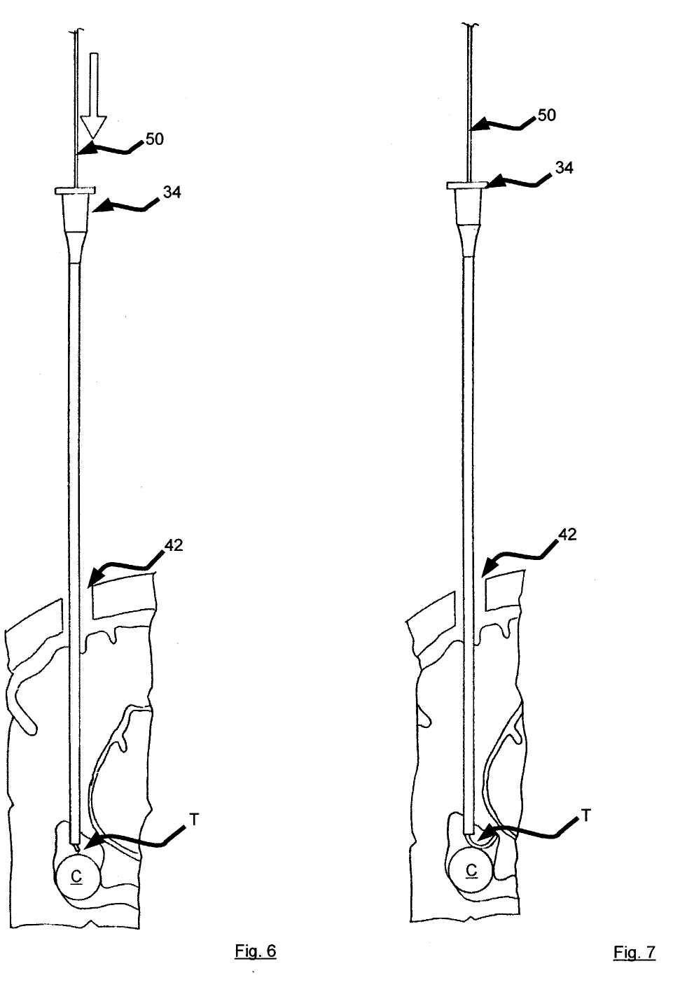

As best seen in FIGS. 6 and 7, wire 50 and bend 54 are thus resiliently bendable such that once bend 54 passes through the distal end of sleeve 34 and comes into contact with clot 45, its end can bend into a partial loop, and once having passed through the distal end of sleeve 34, will provide a visible signal in frame 46, thereby indicating to the surgeon that the wire 50 has reached the target area T. Accordingly, the surgeon thus continues to use the real-time images generated on the CT machine to ascertain where wire 46 should come to a rest inside the ventricle. As best seen in FIG. 7, wire 50 thus comes to a rest with bend 54 formed into partial loop in abutment with clot C.

(Alternatively, wire 46 may not bend but simply come to rest or embed into clot 45. Furthermore, variations on how the function of the loop and/or bend 54 can be achieved are within the scope of the invention and will now occur to those of skill in the art.)

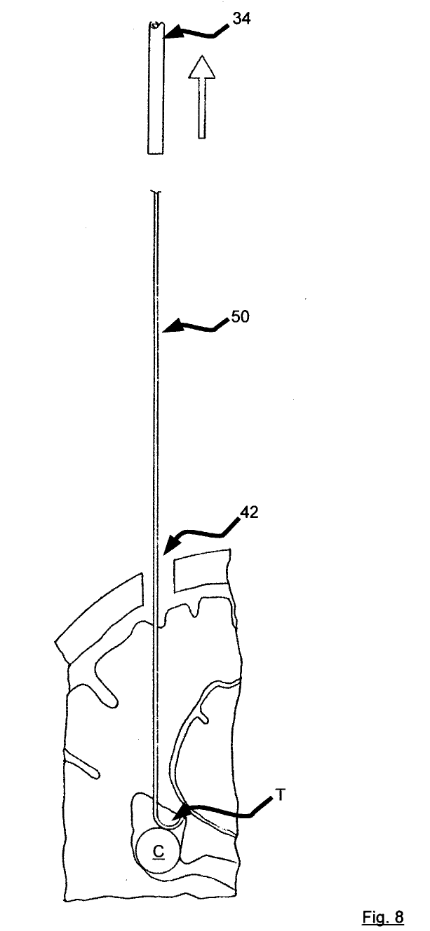

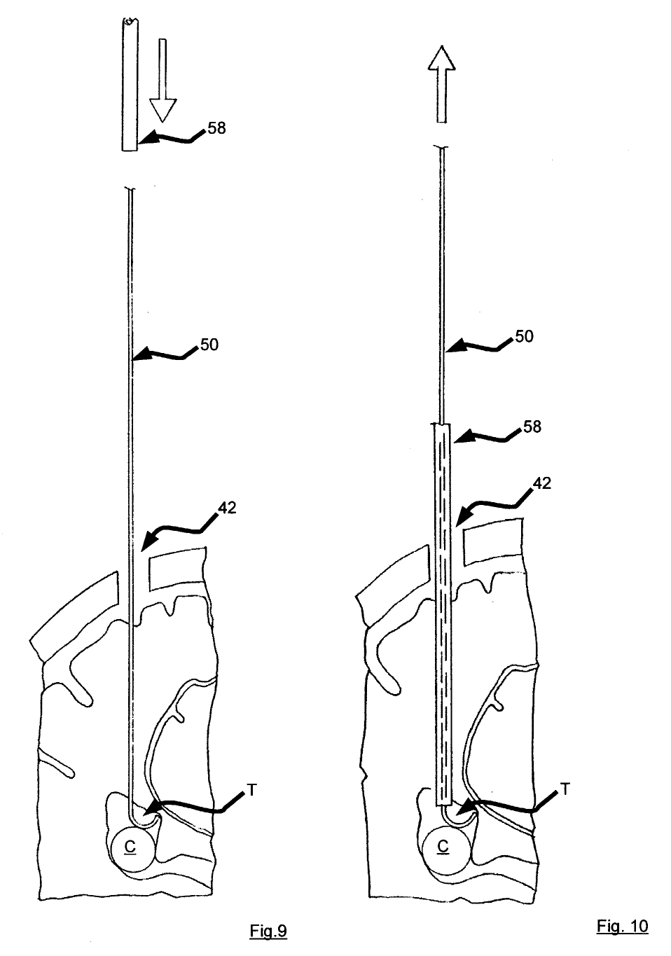

Next, as shown in FIG. 8, sleeve 34 is removed from target area T leaving only wire 40 therein. Next, as shown in FIGS. 9, a catheter 58 is passed over wire 46 towards target area T and clot C using the image of wire 50 shown on frame 46 of the display for guidance in placement of catheter 58. Finally, as shown in FIGS. 10 and 11, wire 46 is removed from head 42 leaving catheter 50 located in the ventricle, in contact with clot 45. Thus, at this point, catheter 50 can be used in any desired manner, such as to drain clot 45 from the ventricle.

It will now thus be apparent that the foregoing can be used to treat hydrocephalus, instead of to remove blood clot C, and that after the final step of the method the inserted catheter can be connected to a complete CSF shunt, or other apparatus. One suitable CSF shunt that could be used is taught in U.S. patent application Ser. No. 09/942,223 entitled “Shunt” and filed on Wednesday, Aug. 29, 2001, for which co-inventor Murphy of the present invention is named as a co-inventor, and the contents of which are incorporated herein by reference.

It should also now be apparent that catheter 58 need not be straight, but could also be bent slightly at its distal end to allow it to be oriented in a desired direction inside the ventricle to effect desired drainage of the ventricle. A suitable bendable wire 50 and catheter 58 combination according to this variation can be purchased from a variety of sources, such as the Bentston-Hanafee-Wilson1 sold by Boston Scientific, One Boston Scientific Place, Natick Mass. 01760, having order number reference 32-160 and Universal Product Number M001321600. Notwithstanding the specificity of the foregoing part number, it is to be further understood, however, that the materials of wire 50 are chosen to reduce beam artifacts, or otherwise substantially preserve the appearance of wire 50 as previously described, and that diameters of catheter 58 and wire 50 will be chosen to suit the particular procedure being performed.

It should now be apparent, however, that the foregoing can be applied to other surgical procedures requiring navigation, as will now occur to those of skill in the art. For example, the teachings herein can be incorporation in conjunction with the teachings of provisional patent application “Kit for Image Guided Surgical Procedures”, bearing application No. 60/366,350 and filed on Mar. 25, 2002 the contents of which are incorporated herein by reference.

In another embodiment of the invention, there is provided a trocar 30a having a sleeve 34a that is convertible into a shunt. Trocar 30a is made from a material that is visible under a real-time imaging beam, such as the Toshiba Acquillon, without producing artifacts that unduly impair navigation. Trocar 30a is comprised of sleeve 34a and stylet 38a.

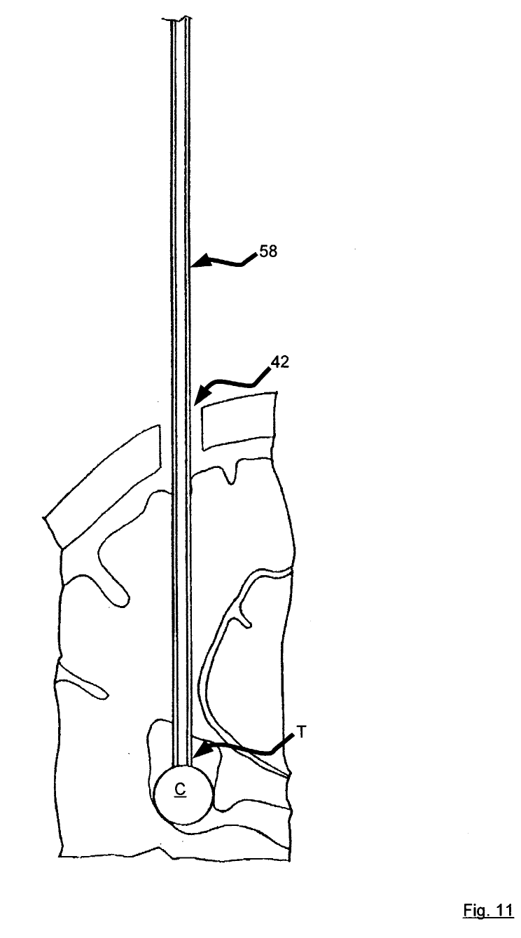

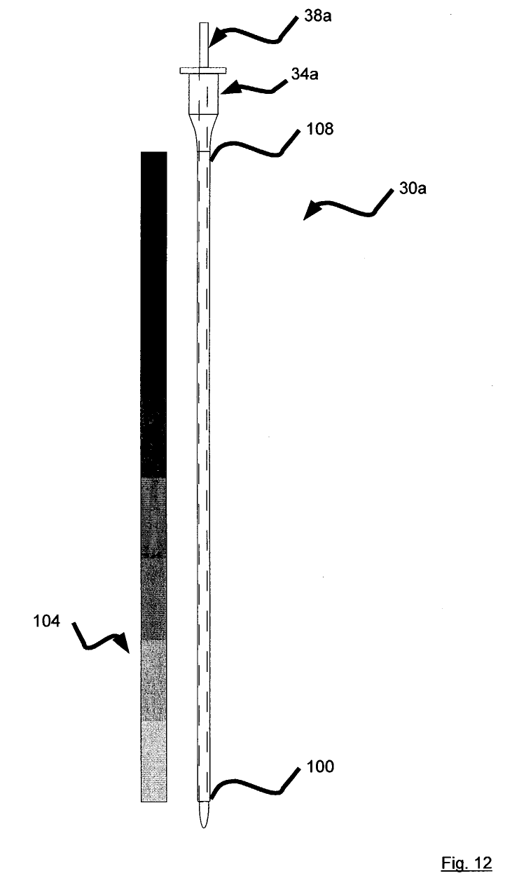

Stylet 38a is made from a rigid material and, when inserted into sleeve 34a, extends past the distal tip 100 of sleeve 34a. The length of sleeve 34a changes in rigidity, being substantially flexible at its distal tip 100, and increasing in rigidity towards its proximal tip 104. Graph 104 shown in FIG. 12 to the left of stylet 38a is shown having a gradient of shading, with light shading at its bottom beside distal tip 100, and becoming increasingly darker shading at its top beside proximal tip 108. The lighter shading represents the flexibility of sleeve 34a, whereas the increasing darker shading represents the increasing rigidity of sleeve 34a. The flexibility of sleeve 34a at distal tip 100 is substantially the same as catheter 58, whereas the flexibility of sleeve 34a at proximal tip 108 is substantially the same as the flexibility of stylet 38a.

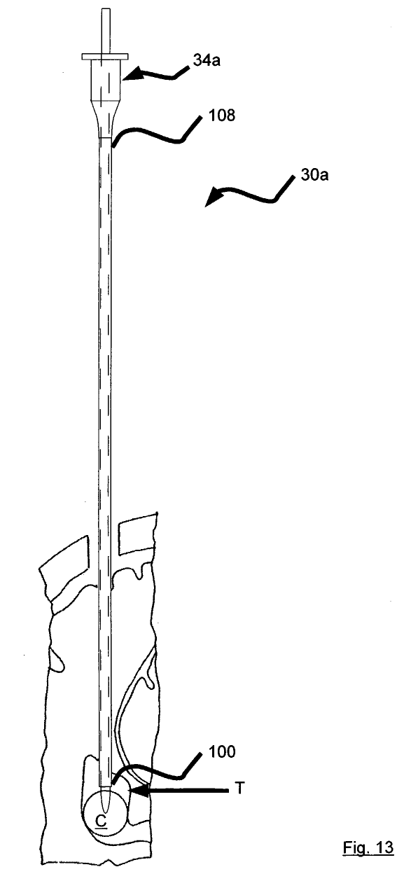

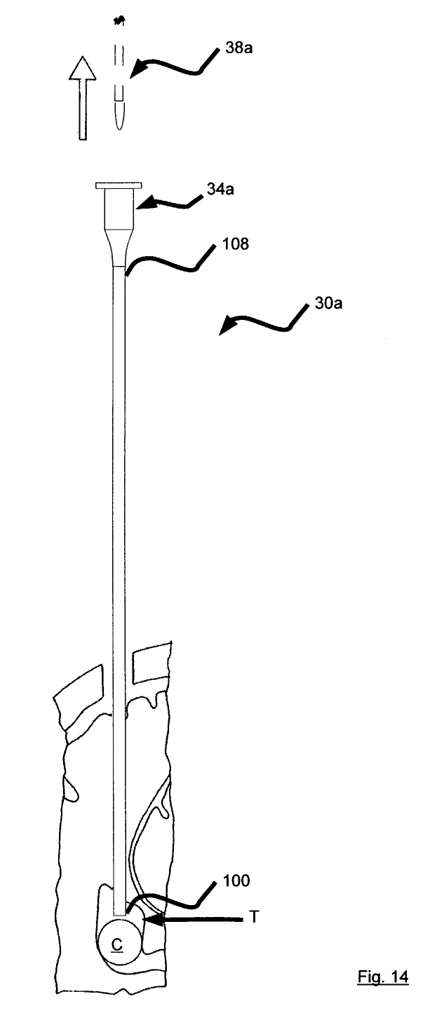

In use, as seen in FIG. 13, trocar 30a is inserted into patient P's head in substantially the same manner as trocar 30 as previously described, under a real-time imaging beam. The rigidity of stylet 38a provides rigidity to flexible tip 100 of sleeve 34a during insertion. Once tip 100 reaches the target area T, then, as shown in FIG. 14, stylet 38a can be removed, leaving tip 100 in target area T. Sleeve 34a can then be used as a catheter, similar to catheter 58 previously-described.

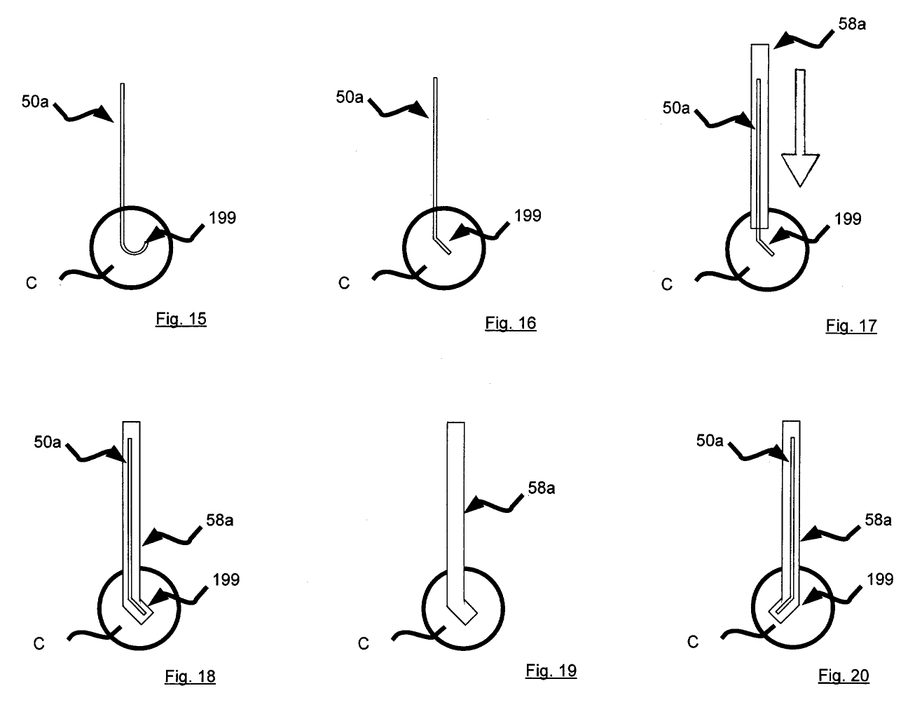

FIGS. 15-19 show another embodiment of the invention varying on the embodiments shown in FIGS. 1-10. In FIG. 15, a wire 50a is inserted into clot C in substantially the same manner that wire 50 was inserted into target area T shown in FIG. 9. In FIG. 15, wire 50a has a looped distal end 199. In FIG. 16, wire 50a is shown to be pulled upwards, (but without removing end 199 from clot C), so that the looped distal end 199 of wire 50a, which is embeddedin clot C, bends into a hockey-stick shape. In FIG. 17, a catheter 58a is passed over wire 50a in substantially the same manner as catheter 58 is passed over wire 50 in FIG. 9. In FIG. 18, catheter 58a is shown substantially covering wire 50a and tip 199, and accordingly, the distal tip of catheter 58a will assume a shape complementary to the hockey-stick shape of end 199. In FIG. 19, wire 50a has been removed, thereby leaving cathether 58 embedded inside clot C with a hockey-stick shaped end that can then be used for treating clot C.

In general, it will now be understood that the steps shown in FIGS. 15-19 can be performed under substantially real-time image guidance, and such steps can be used, or varied, to allow the surgeon to navigate inside clot C (or other target area) and manipulate the end of wire 50a and/or the end of catheter 58a into a desirable position and/or shape so that a desired treatment can be effected on clot C (or other target area). It should now be further apparent that the particular shapes of end 199, and the shapes and directions that end 199 is oriented can be multi-fold. In particular, it should be understood that end 199 can be rotated within clot C (or other target area), and thereby correspondingly allow for greater control over the placement of catheter 58a and the end therof. For example, in FIG. 20 wire 50a and catheter 58a are shown having been rotated one-hundred-and-eighty-degrees within clot C.

While only specific combinations of the various features and components of the present invention have been discussed herein, it will be apparent to those of skill in the art that desired subsets of the disclosed features and components and/or alternative combinations of these features and components can be utilized, as desired. For example, while the embodiments discussed herein refer to navigating and drainage of ventricles, (for treatment of conditions associated therewith, such as intraventricular hemorrhage and the like) it will be understood that other spaces within the head can be navigated and/or drained using the present invention. One space includes the sub-arachanoid space. Another space includes the parenchyma, and the embodiments described herein could be used, for example, to treat conditions such intrapranchymal hematoma in the parenchyma. It is also contemplated that the embodiments herein can be suitably modified for use in navigation of surgical procedures in other areas of the human body, other than those specifically described herein, and that such other modified embodiments are within the scope of the invention.

Furthermore, it is to be understood that the wire 46, in other embodiments, need not be bendable, and can in fact be of a desired rigid material depending on the procedure being performed and/or the trocar being used in the procedure. In general the type of wire chosen, and the method of its insertion, is such that the introduction of the wire is atraumatic, or have an acceptably low risk of causing trauma.

Furthermore, other imaging beams can be used other than CT, such as ultrasound, X-ray, or MRI. In all such imaging beams, the materials chosen to make the various embodiments herein are selected to have sufficient physical strength to and characteristics to pierce the tissue between the exterior of the patient and the chosen target area, while having physical characterstics such that when exposed to their chosen imaging beam, they will be visible to a surgeon, without presenting undue artifacts and thereby allow the surgeon to perform the procedure under substantially real-time image guidance.

Furthermore, where the presently preferred embodiments of the present invention teach a trocar 30 and wire 46 being made from a material resulting in reduced beam artifacts depending on the imaging system used, it is to be understood that, while presently less preferred, in variations of the invention it is possible to use certain currently available trocars 30 and wires 46 that can produce less desirable beam artifacts, but which are nonetheless usable in embodiments of the invention when used by appropriately skilled surgeons and to treat certain conditions that can tolerate somewhat less precise navigation.

It is to be further understood that the types of catheters discussed herein are not particularly limited, and that multi-lumen catheters, and/or catheters with multiple holes around the periphery of their distal end are also within the scope of the invention.

The above-described embodiments of the invention are intended to be examples of the present invention and alterations and modifications may be effected thereto, by those of skill in the art, without departing from the scope of the invention which is defined solely by the claims appended hereto.

We claim:

1. A method for implanting a shunt under guidance of a substantially real-time imaging display system comprising the steps of:
inserting a trocar comprised of a stylet and a sleeve, the trocar having a rigidity to travel to a target area in mammalian tissue and having a radioopacity that substantially preserves the trocar's appearance when the trocar is viewed the system;
removing the stylet from the trocar and leaving the trocar inside the target area;

2. The method of claim 1 further comprising the additional steps of:
inserting a wire into the sleeve having, the wire having a rigidity to travel through the sleeve and a radioopacity that substantially preserves the wire's appearance when the wire is viewed under the system, the insertion terminating until a distal tip thereof resides in the target area; and,
removing the sleeve of the trocar and leaving the wire in the target area.
passing a catheter over the wire until the catheter reaches a desired location in the target area;
removing the wire and leaving the catheter therein.

3. The method of claim 2 further comprising, after the passing step, the additional step of:
manipulating, under the imaging system, the wire and the catheter until the wire and the catheter are in a desired position within the target area.

4. A medical device for use under a guidance of a substantially real-time imaging display system comprising:
a body for travelling through mammallian tissue and having a rigidity to travel therethrough to a target area therein and having a radioopacity that substantially preserves the body's appearance when the body is viewed under the system.

5. The medical device according to claim 4 wherein mammallian tissue is human tissue.

6. The medical device of claim 4 wherein the device is a trocar having a sleeve and a stylet.

7. The medical device according to claim 6 wherein target area is selected from the group consisting of the ventricular system, intraparachymal space, subarachnoid space, subdural space.

8. The medical device according to claim 6 wherein the target area is a clot in the ventricular system.

9. The medical device according to claim 6 wherein the target area is an excess of CSF fluid in the ventricular system

10. The medical device of claim 4 wherein the imaging system is selected from the group consisting of a real-time CT machine, and a real time MR machine.

11. The medical device according to claim 4 wherein the system is the Toshiba Acquillion.

12. The medical device according to claim 4 wherein the body is either coated-with or made-from a material that is hydrophilic.

13. The medical device according to claim 4 wherein the body is either coated-with an infection-resistant material.

14. The medical device of claim 4 wherein the device is a wire for passing down a sleeve resting in the target area.

15. The medical device of claim 14 wherein the wire has a resiliently deformable loop on the end thereof having a straightened position for passing down the sleeve and a looped position for engaging with the target area.

16. A method for implanting a shunt comprising the steps of:
inserting a trocar guided under a substantially real-time imaging system into a target area of the skull;
removing the stylet from the trocar and leaving a sleeve thereof inside the ventricular system;
inserting a radioopaque wire into the sleeve using the display until a distal tip thereof resides in a desired location of the ventricular system;
removing the sleeve and leaving the wire in the ventricular system;
passing a catheter over the wire until the catheter reaches a desired location in the ventricular system; and,
removing the wire and leaving the catheter therein.
